SEQUENCE LISTING (4 sequences)

SEQ ID NO: 1 - DNA, 580 bp, Amblyomma hebraeum
gggagctgcgtcgctttgttccacgttgacctcgaggattcgacgggcaactgcagcaacgcgaacacgagaaagttcgcccgccttgcggctgtggccgagaacctcgtcaataacgtcacccatgttgatcaccaaggacctgatgcagaaaagtacggagaacaaaacgttctgcatcagcatcaacctggccgtcctgaaattcgcaactgatgctggaaaccctggtgaccgctgcgactcggaggacgaggtcgcctactcggaggtgtgccagttgaacagcgcggttccagtgtacgacatgaactggatgaccgcgtcactgagtgacagccggcagttctacacgttcgagaaggctgaaatgctactatctaaggtccttttcctcaaggcgtggttcccgtcgctctgcgtcgccactttccacgccgagcttgacactggtcgatagagaggcctggcccgcattccgatcactacgcccaggctgactcaatgtgcgcaaaggactccaatcctgctgttacatgggcgacgaaacctctgggagaataaacgccctaaaattctc

SEQ ID NO: 2 - DNA, 509 bp, Amblyomma hebraeum
cctcgtgccgaattcggcacgagggatgaatccggaaatcctgcaaagtacgcgaacgaggaaactctcggagtagccagctccgagcagtggctacctgaagatggcgaaacagggacttctgaagaaagtgcgttcaccgctgccgctacagaaggggaagtcgaagccgagggcgctgcagcaaatgaggagccgactagtgcgggggccacaacgggcacttcatcccatggcgaagcagtggctgagcaaggggaaccgcgggctcgacgtagtttcgaagcagctggcgagggtcctggggagcctgcggtgatccacgtcgggacagccgaggcgaatggagcctttgacgccttcaacatggcccggaagatggccgaggcactcttcaacaataagagcagtactcccagcgccgcaaccgggaaggttcccaacttgattgtgcccagcgcagtccaaaagttcgactcctttcctgggcttttggtgataagaaaaag

SEQ ID NO: 3 - protein, 111 aa, Amblyomma hebraeum
MLITKDLMQKSTENKTFCISINLAVLKFATDAGNPGDRRDSEDEVAYSEVCQLNSAVPVYDMNWMTASLSDSRQFYTFEKAEMLLSKVLFLKAWFPSLCVATFHAELDTGR

SEQ ID NO: 4 - protein, 71 aa, Amblyomma hebraeum
MAKQGLLKKVRSPLPLQKGKSKPRALQQMRSRLVRGPQRALHPMAKQWLSKGNRGLDVVSKQLARVLGSLR

(19) United States
(12) Patent Application Publication
(10) Pub. No.: US 2007/0275000 A1
(43) Pub. Date: Nov. 29, 2007

(54) Tick Engorgement Factor Proteins

(76) Inventors: Rueben Kaufman, St. Albert (CA); Brian Weiss, New Haven, CT (US)

Correspondence Address: CRAIG K. SHERBURNE, #236, 6080-88TH STREET, EDMONTON, ALBERTA, T6E 6G4, CA

(21) Appl. No.: 10/571,526

(22) PCT Filed: Sep. 8, 2004
(86) PCT No.: PCT/CA04/01647

§ 371 Date: Apr. 23, 2007

Related U.S. Application Data
(60) Provisional application No. 60/501,415, filed on Sep. 10, 2003.

(51) Int. Cl. A61K 39/00; A61K 38/16; A61K 39/395; A61P 31/00; C07H 21/00; C07K 14/00; C12N 1/00; C12N 15/63; G01N 33/00

(52) U.S. Cl. 424/185.1; 435/243; 435/320.1; 436/86; 514/12; 530/324; 530/350; 530/387.1; 536/23.1

(57) ABSTRACT

The invention provides for novel polynucleotides and associated peptides providing tick Engorgement Factor activity and methods for using same for vaccines, thereby decreasing transmission of tick-borne disease and tick-borne pathogens.

### CROSS-REFERENCE TO RELATED APPLICATION

This application claims benefit of U.S. Provisional Patent Application No. 60/501,415 filed Sep. 10, 2003.

### FIELD OF THE INVENTION

The present invention relates generally to feeding induced proteins from the male reproductive system identified in the tick Amblyomma hebraeum which trigger engorgement in the female tick. More specifically, this invention relates to tick antigens and the nucleic acid sequences which encode them that are useful for conferring tick immunity in a subject and in pharmaceutical compositions and vaccines to elicit an immune response. Also within the scope of this invention is an antibody or an antigen-binding portion thereof that specifically binds a polypeptide of the invention and composition comprising such an antibody or an antigen-binding portion.

### BACKGROUND OF THE INVENTION

Ticks are among the most important vectors of human and animal pathogens including arboviruses, rickettsiae, spirochetes, parasitic protozoa and possibly nematodes. (Sonenshine, D. E. (1993). Biology of Ticks, Volume 2 (Oxford University Press: Oxford)). The incidence of tick borne disease has risen in recent years and is considered to be a major public health problem. Some species of tick secrete a paralytic toxin capable of disabling or killing their host. Furthermore, severe infestations can result in host anaemia, loss of appetite, weakening of the immune system, disruption of liver metabolism and excessive hair loss (Nelson, W. A. et. al. (1977). Interaction of Ectoparasites and Their Hosts. J. Med. Entomol. 13: 389-428).

Ticks are divided into three families: Nuttalliellidae, Ixodidae and Argasidae. The family Nuttalliellidae contains a single species (Nuttalliella namaqua) about which very little is known (Keirans, J. E., et al. (1976). Discovery of Nuttalliella namaqua Bedford (Acarina; Ixodidea; Nuttalliellidae) in Tanzania and redescription of the female based on scanning electron microscopy. Ann. Entomol. Soc. Am. 698: 926-932). Ticks of the family Argasidae have a soft, leathery cuticle and lack a scutum. Argasiq ticks mate off the host, and normally exhibit nidiculous host-seeking behaviour (i.e. they inhabit the nests, caves, burrows, etc. of their host). Adult argasid ticks feed to engorgement within one hour.

Ticks of the family Ixodidae are the most damaging to humans and animals alike. Representative of the Ixodids include the livestock ravaging cattle ticks, Boophlius microplus and Amblyomma hebraeum, the lyme disease transmitting deer tick, Ixodes scapulans, and the typhus and tularaemia transmitting lone star tick, Amblyomma americanum.

One way to prevent Tick infestation is to control the tick population by use of chemicals called acaricides. However, chemical control using acaricides poses significant problems for the environment and public health. In addition, ticks are rapidly developing resistance to the chemicals used, making this approach of poor efficacy in the long term. Finally, acaricides must be applied frequently, making this approach labour intensive.

An alternative method for controlling a tick population is host vaccination. If a host animal is vaccinated against specific tick-derived antigens, tick feeding is inhibited. Tick immunity, therefore, is the capacity of previously exposed hosts to interfere with tick feeding. The results of inhibiting tick feed includes less salvation (thus less pathogen transmission to the host) and less oocyte development.

International Application Number PCT/GB01/01834 teaches the use of tick cement proteins, secreted by the tick salivary glands. In the production of vaccines for protecting animals against the bite of blood-sucking ectoparasites and against the transmission of viruses, bacteria and other pathogens by such ectoparasites.

U.S. Patent Application No. 0010046499 provides 15 novel polypeptides isolated from the salivary glands of Ixodes scapularis useful in eliciting a tick immune response of tick immunity as manifested by one or more of the following: reduction in the duration of tick attachment to a host, reduction in the weight of ticks recovered after detaching from the host as compared to the weight of ticks that attach to non-immune hosts, failure of the ticks to complete their development, and failure to lay the normal number of viable eggs.

Finally International Applicaiton No. PCT/USO1/12189 teaches the use of the proinflammatory cytokine, Macrophage Migration Inhibitory Factor (MMIF), for inducing immunity to ticks, thereby reducing the incidence of tick born infections in animals.

### SUMMARY OF THE INVENTION

The present invention provides novel tick antigens useful for inducing an immune response against tick feeding and egg development. In particular, the present invention relates to the identification and characterization of tick antigens isolated from the tests/vas deferens of fed Amblyomma hebraeum mates. One aspect of the invention provides compositions and methods for conferring tick immunity and for preventing or lessening the transmission of tick borne pathogens. The A. nebraeum polypeptides disclosed herein are particularly useful in single and multicomponent vaccines against tick bites and infections by tick-borne pathogens.

More particularly, this invention provides two novel tick polypeptides, nucleic acid sequences encoding the novel polypeptides and antibodies (or antigen binding portions thereof) specific for the polypeptides. The invention further provides compositions and methods comprising the polypeptides, nucleic acid sequences and antibodies. Finally, the invention further provides a single or multi-component pharmaceutical composition or vaccine comprising one or more tick antigens, preferably one or both of the novel polypeptides, or antibodies of this invention.

In one embodiment, the invention provides two substantially pure polypeptides characterized as having an amino acid sequence as set forth in SEQ ID NO: 3 and SEQ ID NO: 4, respectively. In another embodiment, the invention provides a method for producing the two tick polypeptides. The method includes expressing a polynucleotide encoding one or the other of the invention polypeptides in a host cell and recovering the respective polypeptide.

In a further embodiment, the invention relates to nucleic acid molecules, including DNA, cDNA or RNA sequences that encode the tick polypeptides of the invention. The nucleic acid molecules of the invention include recombinant molecules comprising the nucleic acid molecules of the invention, unicellular hosts transformed with these nucleic acid sequences and molecules, and methods of using those sequences, molecules and host produced tick polypeptides and vaccines comprising them. The nucleic acid molecules of the invention are advantageously used to make probes and polymerase chain reaction primers for use in isolating sequences coding for additional tick antigens. The invention includes polynucleotides encoding the invention polypeptides, as set forth in SEQ ID NO: 1 and SEQ ID NO: 2, respectively. The invention includes polynucleotides encoding the invention polypeptides, as set forth in SEQ ID NO: 1 and SEQ ID NO: 2 in an expression cassette operably linked to a promoter.

In another embodiment, the invention provides an antibody that binds to one or both of the two invention polypeptides or binds to immunoreactive fragments thereof. Such antibodies include polyclonal or monoclonal antibodies.

In yet another embodiment, the invention provides a method for inducing an immune response to a tick polypeptide in a subject, including administering to the subject a pharmaceutical composition containing an immunogenically effective amount of one or both of the polypeptides characterized as having amino acid sequences as set forth in SEQ ID NO: 3 and SEQ ID NO; 4.

Also within the scope of this invention is a method for detecting antibody to the tick polypeptides in a sample comprising contacting the sample with one of the polypeptides in question, or fragments thereof, under conditions which allow the antibody to bind to the tick polypeptide and detecting the binding of the antibody to the tick polypeptide, or fragments thereof.

Finally, this invention also provides methods for the identification and isolation of additional tick polypeptides, as well as compositions and methods comprising such polypeptides.

### BRIEF DESCRIPTION OF THE DRAWINGS

FIG. 1a shows a secondary screen of unfed and fed testis cDNA clones, using a mixed cDNA unfed testis/vas deferens probe and a mixed cDNA fed testis/vas deferens probe, respectively.

FIG. 1b shows PCR-amplification of 35 feeding induced clones, which include the two clones encoding AhEF.

FIG. 2 shows the restriction endonuclease analysis of all constructs to confirm the presence of PCR-amplified feeding-induced clone inserts. All purified constructs were digested to completion using EcoRl and Xhol restriction enzymes and then subjected to electrophoresis on 1.0% agarose gels.

FIG. 3a shows western blots of crude cell lysates containing ᵣAhEFα and ᵣAhEFβ (the expression products of constructs Aht/VD 9 and AhT/VD 22, respectively)

FIG. 3b shows SDS-PAGE of crude lysate (L) and the five 1-ml elutions (E1-E5), stained with coomassie blue. Molecular weight standards are as follows, from top down; 148 kD, 98 kD, 64 kD, 50 kD, 36 kD and 16 kD.

FIG. 4a is a Northern blot analysis of total RNA from fed salivary glands (SG), fed testis/vas deferens (F) and unfed testis/vas deferens(U) when probed with radio-labelled clone AhT/VD 9 PCR product.

FIG. 4b shows a Northern blot of total RNA from fed salivary glands, fed testis/vas deferens(F) and unfed testis/vas deferens(U) when probed with radio-labelled clone AhT/VD 22 PCR product.

FIG. 5 shows the results of the EF bioassay when performed using crude homogenates made from the testis/vas deferens(T/VD) of fed males.

FIG. 6a shows the dose response curve when ticks were injected with various doses of purified ᵣAhEF.

FIG. 6b shows the degree of SG degeneration and ovary development in virgin females that were injected with 0.03-1.0 μg of pure ᵣAhEF.

FIG. 7 shows the effects of ᵣAhEF on egg production in A. hebraeum.

FIG. 8a shows the nucleotide sequence and amino acid sequence of AhT/VD 9 and ᵣAhEFα respectively. The start codon (atg), the stop codon (tag) and polyadenylation signals are shown in bold face.

FIG. 8b shows the nucleotide sequence and amino acid sequence of AhT/VD 22 and ᵣAhEFβ respectively. The start codon (atg), stop codon (tga), polyadenylation signals and the Kozak consensus sequence are shown in bold face.

### DETAILED DESCRIPTION OF THE INVENTION

The present invention discloses two polypeptides isolated from extracts of testis/vas deferens from fed A. hebraeum mates, which together stimulate engagement in co-feeding females. It has been previously shown that male D. variebilis stimulate engorgement in co-feeding females by transferring an “engorgement factor” (EF) to them during copulation. (Pappas and Oliver (1972). Reproduction in Ticks (Acari:Ixodidea). 2 Analysis of the Stimulation for Rapid and Complete Feeding of Female Dermacentor variabilis. J. Med. Entomol. 9: 47-50).

Adult female A. hebraeum require 10 to 14 days to feed to repletion. The feeding cycle consists of three phases:
1. A preparatory feeding phase (1-2 days), during which the female inserts her mouthparts into the host epidermis, established a feeding lesion and secretes a cement like cone to securely attach herself to the skin;
2. A slow feeding phase (7-10 days), during which the female feeds to approximately 10 times her original unfed weight by imbibing blood and other tissue fluids; and
3. A 24-36 hour rapid feeding phase, during which the female increases her weight a further ten-fold, so that at engorgement she weighs approximately 100 times her original unfed weight.

(Balashov, Y. S. “Bloodsucking ticks (Ixodoidea)—vectors of diseases of man and animals”, Misc. Publ. Ent. Soc. Am. 8, pp. 161-376 (1972)).

Following engagement, females detach from the host and begin oviposition approximately 10 days later. Larger species can lay up to 23,000 eggs during a single gonotrophic cycle, after which they die.

In A. hebraeum, the transition weight (i.e. 10 times the unfed weight) between the slow and rapid phases of feeding is called the “critical weight” (CW). The CW is characterized by some marked behavioural and physiological changes (Kaufman, W. R. and Lomas, L. O. (1996). Male factors in ticks: their role in feeding and egg development Invert. Repr. Develop. 30: 191-198). If a virgin or mated female is removed from a host while still below the CW, she: 1. will reattach to a new host if given the opportunity; 2. will not resorb her salivary glands; and 3. will not lay a batch of eggs.

A mated female, on the other hand, if removed from the host having exceeded the CW, will: 1. not resume feeding even if given the opportunity; 2. resorb her salivary glands within four days; 3. lay a batch of eggs, the size of which depends on the amount of blood she consumed before removal; and 4. die.

Recent observations show that approximately 90% to 95% of virgin females do not exceed the CW, even if left on the host for a few weeks. However, if a virgin is forcibly removed from the host when above the CW, she will: 1. not reattach to another host if given the opportunity; 2. resorb her salivary glands within eight days; 3. oviposit a batch of infertile eggs, and 4. die.

Tick salivary glands (SG) serve numerous physiological functions:
(a) during periods of dehydration, ticks are capable of water vapor uptake from the atmosphere. They achieve this by secreting a hygroscopic liquid onto the mouthparts. Sorbed water is than imbibed (Rudolph, D., Knulle, W. (1974). Site and mechanism of water vapor uptake from the atmosphere in ixodid ticks. Nature 249: 84-85);
(b) after establishing a feeding lesion, ixodid ticks secrete a cement-like substance from the SG which hardens into a cone surrounding the hypostome, thus anchoring the mouthparts to the host's skin (Moorhouse, D. E., Tatchell, R. J. (1966). The feeding process of the cattle tick Boophilus microplus (Canestrini): A study in host-parasite relations. Parasitol. 56: 623-632);
(c) the SGs of some species secrete anticoagulants and vasoactive substances which facilitate the process of imbibition (Ribeiro, J. C. (1989). The role of saliva in tick/host interactiosn. Ann. Rev. Entomol. 32: 463-478);
(d) in females, the SGs are responsible for concentrating the nutient portion of the blood meal by excreting excess fluid back into the host (Kaufman, W. R. (1983). The function of tick salivary glands. Current Topics in Vector Research 1: 215-247);
(e) males use saliva as a lubricant to aid transfer of the spermatophore into the female genital tract (Feldman-Muhsam, B., Borut, S. (1970). Copulation in ixodid ticks. J. parasitol. 57: 630-634).

The SGs of female ixodid ticks consists of a pair of elongate, glandular masses of three alveolar types (I, II, III) extending from the anterior of the tick to the single pair of spiracles located posterior to the 4^{th} pair of walking legs (Till, W. M. (1961). A contribution to the anatomy and histology of the brown ear tick, Rhipicephalus appendiculatus Neumann. Mem. Entomol. Soc. S. Africa 6: 1-124).

Upon initiation of feeding, significant ultrastructural, cytological and biochemical changes occur within the gland. These changes include the appearance of features characteristic of fluid transport epithelia (Coons, L. B., Kaufman, W. R. (1988). Evidence that developmental changes in type III acini in the tick Amblyomma hebraeum (Acari:Ixodidae) are initiated by a hemolymph borne factor. Exp. Appl. Acarol. 4: 117-139; Fawcett, D. W., Doxsey, S., Buscher, G (1981), Salivary gland of the tick vector (R. appendiculatus) of East Coast fever. I. Ultrastructure of the type III acinus. Tissue Cell. 13: 209-230), increases in cAMP (Shelby, K. S. et al. (1987). Biochemical differentiation of lone star tick, Amblyomma americanum (L), salivary glands: effects of attachment, feeding and mating, Insect Biochem. 17: 883-890) and Na, K-ATPase activity (Kaufman, W. R. (1976). The influence of various factors on fluid secretion by in vitro salivary glands of ixodid ticks, J. Exp. Biol. 64: 727-742).

Within a few days of dropping off the host, the SGs of female A. hebraeum are resorbed (Harris, R. A. Kaufman, W. R. (1981). Hormonal control of salivary gland degeneration in the ixodod tick Amblyomma hebraeum. J. Insect Physiol. 27: 241-248). This process, which is triggered by a hemolymph-borne substance (‘tick salivary gland degeneration factor’; TSGDF), occurs only in ticks which have fed to above a ‘critical weight’ (CW) of approximately 10× the unfed weight (Harris, R. A. Kaufmann, W. R. (1984). Neural involvement in the control of salivary gland degeneration in the ixodid tick Amblyomma hebraeum. J. Exp. Biol. 109: 281-290; Kaufman, W. R., Lomas, L. O. (1996). “Male factors” in ticks: their role in feeding and egg development. Invert. Repro. and Develop. 30: 191-198). Ticks forcibly removed from a host below the CW do not degenerate their SGs, but instead re-attach and resume feeding if a new host presents itself.

In unfed ticks, SGs have virtually no fluid-secretory ability; salivary fluid secretory competence develops gradually during the slow phase of engorgement (Kaufman, W. R. (1976). The influence of various factors on fluid secretion by in vitro salivary glands of ixodid ticks. J. Exp. Biol. 64: 727-742). As a result, ticks below the CW secrete less saliva than do those during the rapid phase of engorgement and are thus likely to transmit less pathogenic material. In addition, these relatively small ticks lay no eggs, a very significant result in terms of controlling tick populations. If ticks are prevented from feeding beyond the CW, their reproductive success and potential for pathogen transmission are inhibited.

Female salivary gland resorption or degeneration is a process which is triggered by the hormone 20-hydroxyecdysone. Early release of 20-hydroxyecdysone in mated females is stimulated by a male factor protein (MF) produced in the testis/vas deferens portion of the gonads of fed mates. Little MF bio-activity is present in crude gonad homogenates from unfed males and cannot be detected in salivary gland homogenates from fed or unfed males. (Lomas, L. O. and Kaufman, W. R. (1992b). An indirect mechanism by which a protein from the male gonad hastens salivary gland degeneration in the female ixodid tick Amblyommma hebraeum. Arch. Insect Biochem. Physiol. 21: 169-178).

Hence, the difference in salivary gland resorption between mated and virgin females is primarily due to MF, which is passed to the mated female in the spermatophore of the male. MF is not associated with the spermatozoa because spermatozoa separated from other male gonad components on a sucrose density gradient, and injected into large, partially-fed virgin females have no MF-bioactivity (Lomas, L. O. and Kaufman, W. R. (1992a). The influence of a factor from the male genital tract on salivary gland degeneration in the female ixodid tick Amblyommma hebraeum. J. Insect Physiol. 38: 595-601).

Though an exact understanding of the underlying mechanism is not necessary to practice the present invention, it is hypothesized that the “engorgement factor” (EF) and “male factor” (MF) may be the same protein. In the present invention, two novel proteins have been identified which are necessary for EF bio-activity. Since all tick-borne pathogens migrate from the mid gut to the salivary glands and then back into the host only after the tick feeds on a host for a minimum time, a disruption in tick feeding would be useful in reducing transfer of pathogen to host. Therefore, the presence in the blood meal of immune factors such as antibodies and immune cells arising from an immune response elicted by immunization with tick EF results in diminished or absent activity of tick EF in the female; resulting in diminished or absent transmission of one or more of these infectious agents. Thus, the immunization effect of EF in inhibiting the engorgement phase of the ticks would result in there being less salivation, and thus less pathogen transmission to the host, and a marked or complete inhibition of oocyte development. Hence, such anti-tick vaccines would be a desirable method for controlling ticks and controlling the rapid growth of tick populations in areas where they transmit pathogens to humans and domestic animals. Tick borne parasites include Borrelia species that cause Lyme disease, Borrelia ionestari, Borrella anseriana, Borrrelia species that cause relapsing fever, Rickettsia rickettsil, Rickettsia conori, Rickettsia cibirica, Coxiella burnetti, Theileria sp., Francisella tularensis, Ehrlichia species that cause ehrrlichiosis and heart-water disease or related disorders, tick-borne encephalitis virus and related viruses, Colorado Tick Fever orbivirus, Babesia species that cause babesiosis, Anaplasma species that cause anaplasmosis, viruses that cause Crimean-Congo Hemorrhagic Fever, and viruses that cause Kyasanur Forest Disease.

The gene expression in the gonads of fed ticks forms the basis of the present invention. In the present invention, the molecular phenotype of the gonad in the male. A hebraeum is characterized and changes in the gene expression in fed males versus unfed males identified. Thirty-five genes were confirmed to be differentially expressed (up-regulated) in the testis/vas deferens of fed compared to unfed males. Of these thirty-five genes, two were found to express proteins that, in combination, exhibit EF bio-activity.

Thus, in accordance with the present invention, the invention provides two novel A. Hebraeum polypeptides and compositions and methods comprising the polypeptides. More specifically, this invention provides AhEFα polypeptide and AhEFβ polypeptide, which act together as engorgement factor or AhEF. Also within the scope of the invention are polypeptides that are at least 75% homologous in amino acid sequence to the aforementioned AhEFα and AhEFβ polypeptides. In preferred embodiments, the polypeptides are at least 80%, 85%, 90% or 95% homologous in amino acid sequence to the aforementioned polypeptides. In more preferred embodiments, the homologous polypeptides have engorgement factor activities of the above-mentioned polypeptides of the invention.

The invention also includes within its scope fragments of the aforementioned two polypeptides. The term “polypeptide fragment” as it is used herein is defined as a polypeptide that has an amino terminal and/or carboxyl-terminal deletion, but where the remaining amino acid sequence is identical to the corresponding positions in the naturally occurring sequence deduced, for example, from a full length cDNA sequence. Fragments typically are at least 5, 6, 8 or 10 amino acids long, preferably at least 14 amino acids long, more preferably at least 20 amino acids long, usually at least 50 amino acids long and even more preferably at least 70 amino acids long.

The polypeptides of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Polypeptides of invention may also include an initial methionine amino acid residue.

The AhEFα polypeptide sequence is set forth in SEQ ID NO: 3 and the AhEFβ polypeptide sequence is set forth in SEQ ID NO: 4. The present invention further includes conservative variation of SEQ ID NO: 3 and SEQ ID NO: 4. The term “conservative variation” and “substantially similar” as used herein denotes the replacement of an amino acid residue by another, biologically similar residue. Examples of conservative variations include the substitution of one hydrophobic residue such as isoleucine, valine, lysine or methionine for another, or the substitution of one polar residue for another, such as the substitution of one hydrophobic residue such as isoleucine, valine, lysine or methionine for another, or the substitution of one polar residue for another, such as the substitution of arginine for lysine, glutamic acid or aspartic acid, or glutamine for asparagine and the like. The terms “conservative variation” and “substantially similar” also include the use of a substituted amino acid in place of an unsubstituted parent amino acid provided that antibodies raised to the substituted polypeptide also amino react with the unsubstituted polypeptides.

The term “isolated” polypeptide refers to a polypeptide that is substantially free from the proteins and other naturally occurring organic molecules with which it is naturally associated. Purity can be measured by an art known method, e.g., column chromatography, polyacrylamide gel electrophoresis, or HPLC.

An isolated polypeptide may be obtained, for example, by extraction from a natural source (e.g., tick testis/vas deferens), by expression of a recombinant nucleic acid molecule encoding the polypeptide, or by chemical synthesis of the polypeptide. In the context of a polypeptide obtained by extraction from a natural source, “substantially free” means that the polypeptide constitutes at least 60% (e.g., at least 75%, 90%, or 99%) of the dry weight of the preparation. A protein that is chemically synthesized, or produced from a source different from the source from which the protein naturally originates, is defined substantially free from its naturally associated components. Thus, an isolated polypeptide includes recombinant polypeptides synthesized, for example, in vivo, e.g. in the milk of transgenic animals, or in vitro, e.g., in a mammalian cell line, in E. coli or other single celled micro-organism, or in insect cells.

Also included in the invention are polypeptides carrying modifications such as substitutions, small deletions, insertions or inversions, which polypeptides nevertheless have substantially the biological activity of AhEFα or AhEFβ, or the combination of the two. Consequently, included in the invention is the polypeptide, the amino acid sequence of which is at least 95% identical (e.g., at least 96%, 97%, 98%, or 99% identical) to amino acid sequence set forth as SEQ ID NO: 3 or SEQ ID NO: 4 in the sequence listing.

A further embodiment of the invention is polynucleotides, including DNA, cDNA and RNA, encoding the polypeptides of the invention. More specifically, the invention includes two novel DNA molecules encoding the polypeptides of the invention. In particular, the invention provides a DNA molecule comprising the DNA sequence encoding the AhEFα polypeptide and the AhEFβ polypeptide, as set forth in SEQ ID NO: 1 and SEQ ID NO: 2, respectively.

Consequently, the invention provides an isolated nucleic acid molecule encoding either AhEFα or AhEFβ polypeptide, or a conservative variation thereof. An “isolated nucleic acid” is a nucleic acid the structure of which is not identical to that of any naturally occurring nucleic acid or to that of any fragment of a naturally occurring genomic nucleic acid spanning more than three separate genes. The term therefore covers, for example: (a) a DNA which has the sequence of part of the naturally occurring genomic DNA molecule but is not flanked by both of the coding sequences that flank that part of the molecule in the genome of the organism in which a naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleic acid sequence that is part of a hybrid gene, i.e. a gene encoding a fusion protein.

The nucleic acid molecules of the invention are not limited strictly to molecules including the sequences set forth as SEQ ID NO: 1 and SEQ ID NO: 2. Rather, the invention encompasses nucleic acid molecules carrying modifications such as substitutions, small deletions, insertions, or inversions, which nevertheless encode proteins having substantially the biological activity of the AhEFα and AhEFβ polypeptide according the invention, and/or which can serve as hybridization probes for identifying a nucleic acid with one of the disclosed sequences.

Included in the invention are nucleic acid molecules, the nucleotide sequence of which is at least 95% identical (e.g., at least 96%, 97%, 98%, or 99% identical) to the nucleotide sequence shown as SEQ ID NO: 1 and SEQ ID NO: 2. The determination of percent identity or homology between two sequences is accomplished using the algorithm of Karlen and Altschul (1990) Proc. Nat'l. Acad. Sci. USA 87: 2264-2268, modified as in Karlen and Altschul (1993) Proc. Nat'l Acad. Sci. USA 90: 5873-5877. Such an algorithm is incorporated in the NBLAST and XBLAST programs of Altschul et al. (1990) J. Mol. Biol. 215: 403-410. BLAST nucleotide searches are performed with the NBLAST program, score equate 100, word length equals 12 to obtain nucleotide sequences homologous to the nucleic acid molecules of the invention. BLAST protein searches are performed with the XBLAST program, score equals 50, word length equals 3 to obtain amino acid sequences homologous to the protein molecules of the invention. To obtain gapped alignments for comparison purposes, GAPPED BLAST is utilized as described in Altschul et. al. (1997, Nucleic Acids Res. 25: 3389-3402). When utilizing BLAST and GAPPED BLAST programs, the default parameters of the respected programs (e.g. XBLAST and NBLAST) are used.

The term “stringent hybridization conditions” is known in the art from standard protocols (e.g., Current Protocols in Molecular Biology, Editors F. Ausubel et al., John Wiley & Sons, Inc. 1994) and is to be understood as conditions as stringent as those defined by the following: hybridization to filter-bound DNA in 0.5M NaHPO₄ (pH 7.2) 7% sodium dodecyl sulphate (SDS), 1 mM EDTA at plus 65° C., and washing in 0.1×SSC/0.1% SDS at plus 68° C.

Also included in the invention is a nucleic acid molecule that has a nucleotide sequence which is a degenerate variant of nucleic acid disclosed herein, e.g. SEQ ID NO: b 1 and SEQ ID NO: 2. A sequential group of three nucleotides, a “codon”, encodes one amino acid. Since there are 64 possible codons, but only 20 natural amino acids, most amino acids are encoded by more than one codon. This natural “degeneracy” or “redundancy” of the genetic code is well known in the art. It will thus be appreciated that the nucleic acid sequences shown in the sequence listing provide only an example within a large but definite group of nucleic acid sequences that will encode the polypeptides as described above.

In yet another embodiment, this invention provides antibodies or an antigen binding portion thereof, that specifically bind a polypeptide of this invention, and pharmaceutically effective compositions and methods comprising those antibodies. The antibodies of this invention are those that are reactive with a tick feeding induced polypeptide, preferably an A. hebraeum polypeptidse of this invention. Such antibodies may be used in a variety of applications, including detecting expression of tick feeding induced antigens, preferably. A. hebraeum antigens, to screen for expression of novel tick polypeptides, to purify novel tick polypeptides and to confer tick immunity. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complimentary determining region (CDR) fragments, single chain antibodies (scFv), chimeric antibodies, diabodies and polypeptides that contain at least a portion of an immunoglobulin that is sufficient to confer specific antigen binding to the polypeptide.

In a further embodiment of this invention, methods are provided for inducing tick immunity in a host by administering one or more tick polypeptides, preferably. A. hebraeum polypeptides or one or more antibodies of the invention. In particular, a method is provided for preventing or reducing the transmission of tick borne pathogens by administering polypeptides or antibodies of this invention that are effective to induce tick immunity.

The A. hebraeum polypeptides disclosed herein are particularly useful in single and multicomponent vaccines against tick bites and infections by tick-borne pathogens. In a preferred embodiment, the vaccines comprise AhEFα polypeptide AhEFβ polypeptide, or a mixture of AhEFα and AhEFβ polypeptides. Multicomponent vaccines may further comprise polypeptides that characterize other vaccines useful for immunization against tick-borne pathogens.

The preferred compositions and methods of the present invention comprise AhEFα and AhEFβ polypeptides having enhanced immunogenicity. Such polypeptides may result when the native forms of the polypeptides or fragments thereof are modified or subjected to treatments to enhance their immunogenic character in the intended recipient. Examples of ways to enhance immunogenicity of the polypeptides of the present invention are coupling the polypeptides to dinitropherol groups or arsanilic acid, or by denaturation by heat and/or SDS.

Vaccines may further comprise immunogenic carriers such as keyhole limpet hemocyanin (KLH), albumins such as bovine serum albumin (BSA) and ovalbumin, red blood cells, agarose beads and the like.

Any of the polypeptides of the present invention may be used in the form of a pharmaceutically acceptable salt. Suitable acids and bases which are capable of forming salts with the polypeptides of the present invention are well-known to those skilled in the art, and include inorganic and organic acids and bases.

The antibodies of the invention can be used in any subject in which it is desirable to administer in vitro or in vivo immunodiagnosis or immunotherapy. The antibodies of the invention are suited for use, for example, in immunoassays in which they can utilized in liquid phase or bound to a solid phase carrier. In addition, the antibodies in these immunoassays can be detachably labelled in various ways. Examples of types of immunoassays which can utilize antibodies of the invention are competitive and non-competitive immunoassays in either a direct or indirect format. Examples of such immunoassays are enzyme-linked immunoassay (ELISA), radioimmunoassay (RIA) and the sandwich (immunometric) assay. Detection of antigens using the antibodies of the invention can be done utilizing immunoassays which are run in either the forward, reserve, or simultaneous modes, including immunohistochemical assay on physiological samples. Those skilled in the art will know, or can readily discern, other immunoassay formats without undue experimentation.

The invention also provides for monoclonal antibodies which are made from antigens containing fragments of the proteins herein by methods well known to those skilled in the art (Kohler and Milstein, Nature 256: 495 (1975): Coligan et. al. Sections 2.5.1-2.6.7; and Harlow et al., Antibodies: A Laboratory Manual, page 728 (Cold Spring Harbour Pub. 1988), which are hereby incorporated by reference. Briefly, monoclonal antibodies can be obtained by injecting mice with a composition comprising an antigen/ligand, verifying the presence of antibody production by analysing a serum sample, removing the spleen to obtain B lymphocytes, using lymphocytes with myeloma cells to produce hydbridromas, cloning the hybridomas, selecting positive clones that produce antibodies to the antigen, and isolating the antibodies from the hybridoma cultures. Monoclonal antibodies can be isolated and purified from hybridoma cultures by a variety of well established techniques. Such isolation techniques include affinity chromatography with Protein-A Separose, size-exclusion chromatography, and ion-exchange chromatography. See e.g., Coligan et al., sections 2.7.1-2.7.2 and sections 2.9.1-2.9.3; Barnes et al., “Purification of immunogobulin G (IgG)” and “Methods in Molecular Biology”, Vol. 10, pages 75-104 (Humana Press 1992).

Another embodiment of the present invention is a method for treating an animal with a therapeutically effective amount of a tick polypeptide, preferably AhEFα and AhEFβ polypeptides, or a fusion protein or a multimetric protein comprising AhEFα and AhEFβ polypeptides, in a manner to confer tick immunity or prevent or lessen the severity, for some period of time, of infection by tick-borne pathogens.

### EXAMPLE 1

### Isolation and Characterization of Genes Differentially Expressed in the Testis Vas Deferens of Male Amblyomma hebraeum

Ticks. Male A. hebraeum were taken from a laboratory colony maintained in the dark at 26° C. and at a relative humidity of >95%. To allow for sufficient tissue maturation (testis vas deferens (T/VD), accessory gland (AG), salivary glands (SG), gut, synganglia (SYN) and Malphigian tubules (Mt), 30 male ticks were fed per rabbit for >4 days in a foam and cloth backpack as described by Kaufman and Phillips (1973). Ion and water balance in the ixodid tick, Dermacentor andersoni. I. Routes of ion and water excretion. J. Exp. Biol. 58: 523-536, incorporated herein by reference. A total of 2500 male ticks were used.

Tissue/RNA isolation. Males were stuck ventral surface down to a petri dish using a cyanoacrylate glue (Loctite™, Rocky Hill, N.J.), flooded with DEPC treated water and the T/VD, AG, SG, Malphigian tubules (Mt), synganglion (SYN) and gut were dissected out. Tissues were frozen immediatgely on dry ice. Total cellular RNA was extracted by grinding tissues with a mortar and pestle and then further homogenizing in a glass tissue homogeniser in the presence of TRizol™ reagent (Gibco-BRL, Rockville, Md.). Poly (A)+RNA was extract ed using an Oligotex™ mRNA mini kit (Qiagen, Carlsbad, Calif.) according to the manufacturer's protocol.

cDNA library construction. A cDNA library was constructed from 4 μg fed tick T/VD poly (A)+RNA's using a Uni-ZAP XR™ cDNA library synthesis kit and the Gigapack II Gold Packaging Extract (Strategene, La Jolla, Calif.) according to the manufacturer's protocol. The Fed-T/VD library contained between 1×10⁶ to 2×10⁶ independent cDNA clones. Twenty randomly chosen clones were amplified by polymerase chain reaction (PCR), and then were subjected to electrophoresis on a 1% agarose gel for 2 h at 80 volts. The gel was stained with ethidium bromide and viewed over UV light to determine average insert size.

Preparation of DNA probes. Poly (A)+RNA was prepared from fed and unfed testis as described above. One microgram of mRNA, was reverse transcribed using a Timesaver™ cDNA synthesis kit (Amersham Pharmacia, Piscataway, N.J.) to produce a mixed population of double-stranded cDNA probe representative of the mRNA population in each of the tissues. Insert DNA from selected clones were prepared by PCR amplification as described below in the section ‘PCR and secondary screening’.

Probes for all experiments were labelled using random primers and a mixture of dNTP's and Klenow fragment (Random Primers DNA Labelling System; Gibco-BRL, Rockville, Md.). Probes made for the primary and secondary differential screens were triple-labelled ([³²P]αdATP, [³²P] αdCTP and [³²P] αdGTP) while those made for Northern and Southern blots were single labelled ([³²P] αdCTP). Unincorporated nucleotides from each reaction were removed by Sephadex™ G-50 chromatography.

Differential cross-screening of fed T/VD cDNA library. The library was screened unamplified. Differential screening was performed as described by Benton, W. D. and Davis, R. W. (1977). Screening lambda gt recombinant clones by hybridization to single plaques in situ. Science 196: 180-182, incorporated herein by reference. Clones from the fed-T/VD library, using XL1-Blue E. coli cells as a host, were plated at a density of 1500 pfu/150 mm plate. Nylon colony plaque screen hybridization transfer membranes were marked for later re-orientation with plates and screened as defined by the manufacturer (NEN-Dupont, Boston, Mass.). The first of each duplicate set of plaque lifts was screened with [³²P]-labelled fed-T/VD mixed cDNA probe and the second with [³²P]-labelled unfed-T/VD mixed cDNA probe. Lifts were hybridized with the respective T/VD cDNA probe and processed under stringent conditions (final wash with 0.1×SSC/0.1% SDS for 10 min at 65° C.) in Hybridol™ II (Intergen Co., Purchase, N.Y.) Screened blots were exposed for 1-3 days at −70° C. to Kodak X-O Mat film. Unless otherwise noted these conditions were used for all hybridization experiments performed. In the case of the library screening, plaques with different intensities of hybridization signal between the two probes were identified and isolated (Sambrook, J., Fritsch, E. F., Maniatus, T. (1989). Moleculer cloning: a laboratory manual, 2^{nd} ed. Cold Springs Harbor University Press, Cold Springs Harbor, N.Y., incorporated herein by reference).

PCR and secondary screening. PCR was performed on all putative feeding-induced clones isolated after primary screening. A 5 μl sample of each plaque was added to a 95 μl reaction mixture containing ddH₂0, dNTP's (200 μM), PCR buffer (200 mM Tris-HCl (pH 8.4), 500 mM KCl, 50 mM MgCl₂), T3 primer (0.5 μM; 5′-ATT AAC CCT CAC TAA AGG GA-3′), T7 primer (0.5 μM; 5′-TAA TAC GAC TCA CTA TAG GG-3′; BioServe, USA) and 10 units of Taq DNA polymerase. PCR was conducted using an Eppendorf (Westbury, N.Y.) thermal cycler. The amplification program consisted of a three min hotstart at 94° C. followed by 30 cycles at 94° C. for 1 min (DNA denaturation), 50° C. for 1 min (annealing of primers), 72° C. for 3.5 min (DNA elongation) and a final elongation/extension at 72° C. for 7 min. Amplified products were verified by agarose gel electrophoresis.

For secondary screening 0.2 μl of PCR product from each putative feeding-induced clone isolated after primary screening was arrayed onto three gridded nylon membranes (secondary blot). Each membrane was then allowed to hybridized with either [³²P]-labelled fed-T/VD mixed cDA probe or [³²P]-labelled unfed-T/VD mixed cDNA probe. Pre-hybridization, hybridization, wash conditions and the final processing of the blots for the secondary screen were the same as those used for the primary screen.

Analysis of the primary differential screen of 15,000 clones on duplicate plaque lifts, using [³²P]-labelled fed-T/VD cDNA as probe on the first lift and [³²P]-labelled unfed-T/VD cDNA as probe on the duplicate plaque lift, allowed the isolation of 247 clones which apparently displayed higher levels of hybridization with fed testis compared to unfed testis probe (results not shown). Analysis of the secondary screen confirmed 35 putative differentially expressed sequences.

Sequencing and sequence analysis. cDNA clones which passed the secondary screening process were purified using either the QIAquick™ Gel extraction kit or the QIAquick™ PCR purification kit (Qiagen, Mississauga, Ontario). Clones isolated from the secondary screen were submitted to single pass sequencing using a DYEnamic™ ET terminator cycle sequencing premix kit (Amersham Pharmacia, Piscataway, N.J.) in order to generate an expressed sequence tag for each gene in question. Sequenced inserts were run on a PE Applied, Biosystems 377 automated sequencer. Sequence data were analyzed using Genetool™ (Biotolls Inc., Edmonton, Canada) and comparisons with the Genbank database performed by BLAST search (http://www.ncbi.nim.nih.gov/BLAST/).

Northern blots. Three micrograms of total RNA was subjected to electrophoresis on an agarose gel and transferred overnight to Genescreen Plus nylon membranes (NEN-Dupont, Boston, Mass.) following the protocol of Sambrook et al. (Sambrook, J., Fritsch, E. F., Maniatus, T. (1989). Molecular cloning: a laboratory manual, 2^{nd} ed. Cold Springs Harbor University Press, Cold Springs Harbor, N.Y.). Blots were screened with the relevant radio-labeled probe under stringent conditions (as described for the library screens) and then exposed to Kodak X-O Mat film between two intensifying screens.

The intensity of bands on autoradiographs were quantified using the Kodak Digital Science ID image analysis system (Eastman Kodak Co., Rochester, N.Y.). In order to normalize the band intensities to possible variations in RNA loading, we also quantified the relative level of 18S RNA in each lane of the gel used to generate the Northern blot analyzed. The normalized value of any transcript is the intensity of the corresponding band on the autoradiograph divided by the intensity of the 18S RNA band in the photograph of the corresponding sample in the original agarose gel photograph (Coorrea-Rotter, R., Mariash, C., Rosenberg, M. (1992). Loading and transfer control for northern hybridization. BioTechniques 12: 154-158). Statistical analysis was performed using Microsoft Excel software (Microsoft, Wash.).

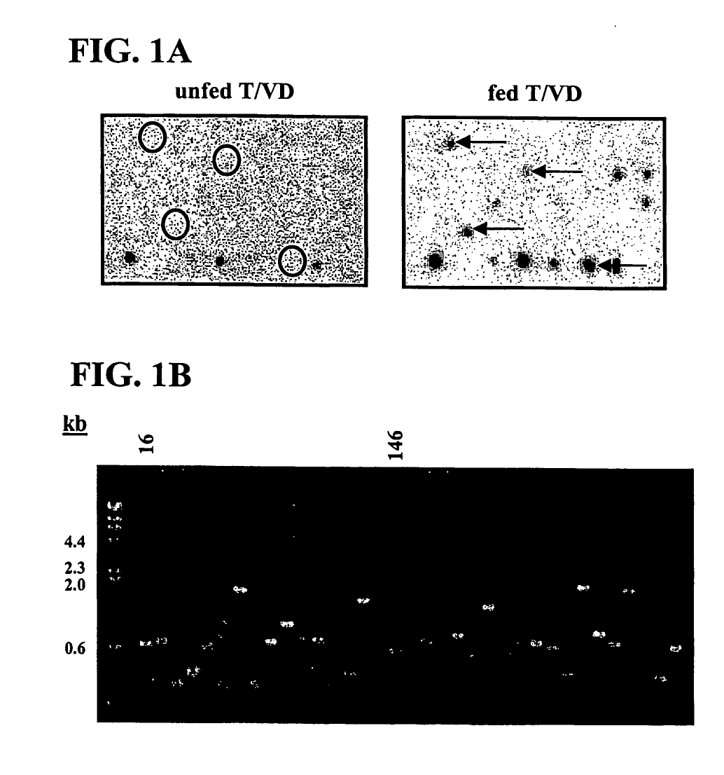

FIG. 1a shows secondary screening of fed testis cDNA clones. Each PCR-amplified cDNA clones isolated from the primary screen (not shown) was spotted onto two nylon membranes. The first membrane was screened with a mix of unfed T/VD probe and the second with a mixed fed T/VD cDNA probe. Clones up-regulated by feeding were then isolated. A total of 35 up-regulated genes were cloned and isolated. FIG. 1b shows the PCR-amplification of the 35 feeding induced clone inserts following the secondary differential screen. Amplified products were electrophoresed on a 1.2% agarose gel at 80 volts for 2 h.

### EXAMPLE 2

### Construct Design and Preparation

Prior to experimentation, all constructs used in this study were drafted using the computer program Gene Construction Kit 2 (Sci Quest Inc., Research Park, N.C.). All PCR primers, designed used Genetool software (Biotools Inc., Edmonton, Canada), were engineered with 5′-EcoRI and 3′-Xhol restriction endonuclease cut sites (Invitrogen Co., Carlsbad, Calif.). Aht/VD 9-1, 5′-GGG AAT TCG GGA TGT TGA TCA CCA AGG ACC TGA-3′; AhT/VD 9-2, 5′-GGC TCG AGG GTC GAC CAG TGT CAA GCT CGG-3′ and Aht/VD 22-1, 5′-GGG AAT TCG GGA TGG CGA AAC AGG GAC TT-3′; AhT/VD 22-2, 5′-GGC TCG AGG GCC GCA GGC TCC CCA-3′.

PCR of cDNA inserts. PCR was performed on all clones containing inserts having complete open reading frames (28 of the 35 clones up-regulated by feeding). A 6-μl sample of each plaque was added to a 95-μl reaction mixture containing ddH₂0, dNTP's (200 μM), PCR buffer (200 mM Tris-HCl (pH 8.4), 500 mM KCl, 50 mM MgCl₂), the appropriate above-mentioned PCR primers (0.5 μM) and 10 units of a combination of Taq and Pfu (10:1) enzymes. PCR was conducted using an Eppendorf (Westbury, N.Y.) thermal cycler. The amplification program consisted of a 3-min hotstart at 94° C., followed by 30 cycles at 94° C. for 1 min (DNA denaturation), 50° C. for 1 min (annealing of primers), 72° C. for 2.5 min (DNA elongation) and a final elongation/extension at 72° C. for 7 min. Amplified products were verified by agarose gel electrophoresis, and appropriately sized bands extracted using a Qiagen gel extraction kit according to the manufacturers protocol.

Cloning. Basic cloning protocols are modified from Ausubel, F. M., Brent, R., Kingston, R. E., Moore, D. D., Seidman, J. G., Smith, J. A., Struhl, K. (1994). Current Protocols in Molecular Biology. (Wiley Interscience, New York). Five microlitres (−1 μg) of purified insert and vector DNA (plB/V5-His or plB/His C; from the InsectSelect™ kit, Invitrogen Co.) were added to separate 40-μl restriction reactions containing 5 μl of 10×restriction buffer, 1 μl (10 U) of EcoRl and Xhol restriction endonuclease (Gibco-BRL, Rockville, Md.) and 33 μl of ddH₂0. Following a 2 h incubation at 37° C., samples were electrophoresed on a 1% agarose gel and bands extracted as mentioned above. Ligation reactions (10 μl) were set up containing the following reagents: 3 μl digested insert DNA, 1 μl digested vector DNA, 5 μl 2×ligation buffer and 1 μl T4 DNA ligase (3 Weiss U; Gibco-BRL). Reactions were incubated for 1 h at room temperature (or overnight at 4° C.).

Constructs were propagated in DH5α competent cells (Gibco-BRL). Between 1-3 μl of each ligation reaction were added to a 50-μl aliquot of DH5α competent cells. Reactions were incubated on ice for 30 min. heat-shocked for 20 s at 37° C. and returned to ice for 2 min. S.O.C. medium (Gibco-BRL; 950 μl) was added to each reaction mixture. Reactions were placed in a shaking incubator at 37° C. for 1 hr at 225 rpm.

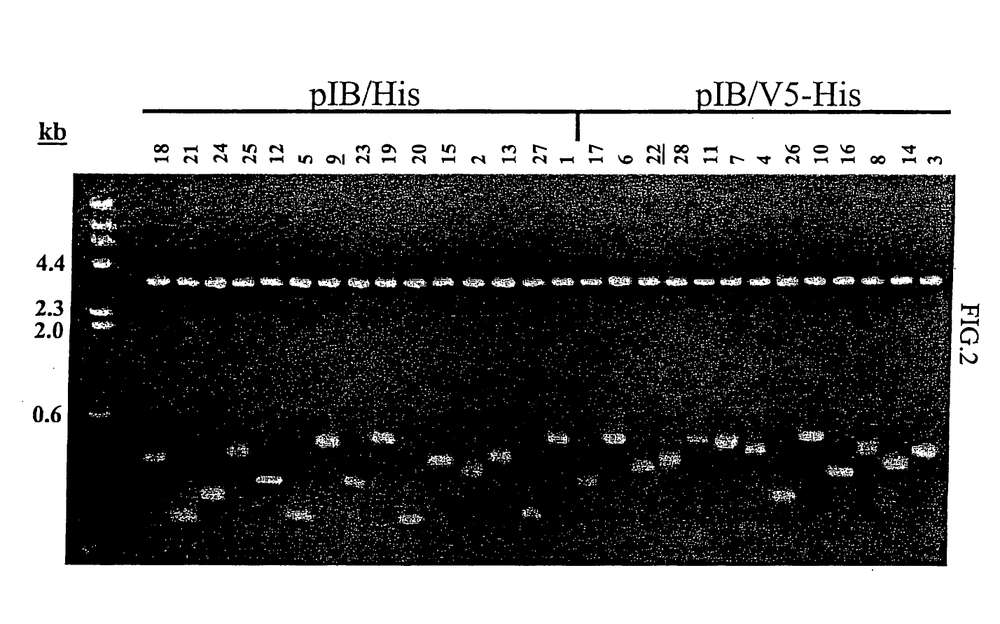

Propagated plasmid constructs were actuated using a Qiagen plasmid mini-prep kit according to the manufacturer's protocol. All purified plasmids were subjected to EcoRl and Xhol restriction endonuclease digestion followed by electrophoresis on 1% agarose gels to verify the presence of insert and vector DNA (see FIG. 2).

Sequencing and sequence analysis. All propagated plasmids were sequenced using a DYEnamic™ ET terminator cycle sequencing premix kit (Amersham Pharmacia, Piscataway, N.J.). Sequencing reaction products were run on a PE Applied Biosystems 377 automated sequencer. Sequence data were analyzed using Genetool and Chromatool™ software (Biotolls Inc., Edmonton, Canada) to confirm that all inserts were ligated into the vector in the proper open reading frame (ORF).

### EXAMPLE 3

### Production and Detection of Proteins from Feeding-Induced T/VD Genes

Transfections. Sf21 cells were maintained in culture prior to transfections. At time of transfection, cells were plated at 60-80% confluency in 60 mm cell culture dishes and left undisturbed for 30 min to allow adhesion to the dish.

Liposome/DNA complexes were all formed in serum-free medium according to the manufacturer's protocol (Invitrogen Co.). Briefly, 1 μg (−10 μl) of purified plasmid DNA (construct containing the gene of interest), and 7.5 μl of Celifectin reagent, were each diluted into separate 100-μl aliquots of serum-free medium (Sf-900 II serum-free medium (SFM); Gibco-BRL) and allowed to stand for −10 min at room temperature. The contents of both tubes were then mixed together and incubated at room temperature for −20 minutes. Positive (plB/V5-His CAT) and negative (no liposome) control transfections were also performed. Sf-900 II SFM (800 μl) was added to each tube containing newly formed liposome/DNA complexes. Each dish of cells was washed with 2 ml of Sf-900 II serum-free medium and gently overlayed with liposome/DNA complex. Dishes were incubated for 7-10 h at 27° C. Following the incubation, the transfection solution was removed and replaced with 2 ml of serum containing cell culture medium. All dishes containing transfected cells were placed in an airtight plastic bag containing moist paper towel to inhibit evaporation.

Detection of proteins. Expression products were harvested 48 h post-transfection. Medium from each transfection dish was frozen at −80° C. to assay for secreted proteins by Western blot analysis. Cell lysis buffer (100 μl; 50 mM Tris pH 7.8, 150 mM NaCl, 1% (v/v) Igepal CA-630) was repeatedly streamed over cells until all were sloughed from bottom of the dish.

Complete lysis was assured by vortexing rapidly for 15 s, and cellular debris was pelleted at 10,000×g for 15 min at 4° C.

Protein concentration of culture medium and cell lysis supernatant was determined by a Branford assay (Bradford, M. M. (1976). A rapid and sensitive method for the quantification of microgram quantities of protein utilizing the principle of protein dye binding. Anal. Biochem. 72: 248-254) using bovine serum albumin as standard. Lysate containing 30 μg of protein was combined with 4×SDS sample buffer (125 mM Tris-HCl pH 6.8, 4% SDS, 50% glycerol, 0.02% bromophenol blue, Sigma) and heated at 95° C. for 5 min. Samples were electrophoresed in 1×SDS running buffer (25 mM Tris, 192 mM glycine, 0.1% (w/v) SDS, pH 8.3) for approximately 90 min through 3% stacked, 12% continuous separating polyacrylamide gels. Protein bands were visualized by staining the gels for 2-24 h with coomassie brilliant blue (Sigma, St. Louis, Mo.) dissolved in 40% methanol/10% acetic acid.

Recombinant protein production was confirmed by Western blot analysis. Proteins were electrophoresed as described above. Polyacrylamide gels and 0.2 μm nitrocellulose membranes (BioRad, Hercules, Calif.) were equilibrated in transfer buffer (25 mM Tris-HCl, 192 mM Glycine, 20% (w/v) methanol, pH 8.3) for 5 min. Proteins were blotted onto the membranes at 100V for 1 h, and protein transfer was confirmed by reversible staining with Ponceau S (Sigma). Following protein visualization, Ponceau S stain was removed by washing blots with milli-Q water. Nitrocellulose membranes were incubated in blocking buffer (50 mM Tris-HCl pH 8.0, 150 mM NaCl, 3% (w/v) ovalbumin, 0.1% (v/v) Triton X-100, 0.1% (w/v) NaN₃) for 30 min at room temperature. Old blocking buffer was removed and the membrane was covered with anti-6×histidine antibody (diluted at 1:3000 in fresh blocking buffer). Nitrocellulose membranes were incubated on a rocking platform for 2 h at room temperature, or overnight at 4° C.

Protein bands were visualized using a goat anti-mouse secondary antibody conjugated to an IRDye 800 (a near-infrared florophore). Following the removal of anti-6×histidine primary antibody solution by washing 4×15 Min in Tween-20/Tns-buffered saline (TTBS: 0.1% Tween-20 in 100 mM Tris-HCl, 0.9% NaCl, pH 7.5), nitrocellulose membranes were again blocked in 10 ml blocking buffer for 20 min. Fluorescently-labelled secondary antibody was then diluted 1:2500 in blocking buffer and added to the nitrocellulose membrane. Following a 1-h incubation at room temperature on a rocking platform, non-bound secondary antibody was removed by washing 4× with TTBS (incubation with secondary antibody and all subsequent wash steps were performed in the dark). Protein bands were visualized using a LI-COR Odyssey infrared imaging system.

FIG. 2 shows the restriction endonuclease analysis of all constructs to confirm the presence of PCR-amplified feeding-induced clone inserts. All purified constructs were digested to completion using EcoRl and Xhol restriction enzymes and then subjected to electrophoresis on 1.0% agarose gels. The first 15 inserts were cloned into the PlB/His C expression vector and the remaining 13 into the plB/V5-His expression vector (which incorporates the 6×histidine detection tag on the opposite end of the ᵣprotein). The continuous line of bands across the gel at −3540 kd represent vector DNA and the variably-sized bands (ranging from 211-540 kB) at the bottom of the gel represent construct inserts. The two constructs (AhT/VD 9 and AhT/VD 22, respectively) containing inserts coding for the proteins having EF bio-activity are underlined.

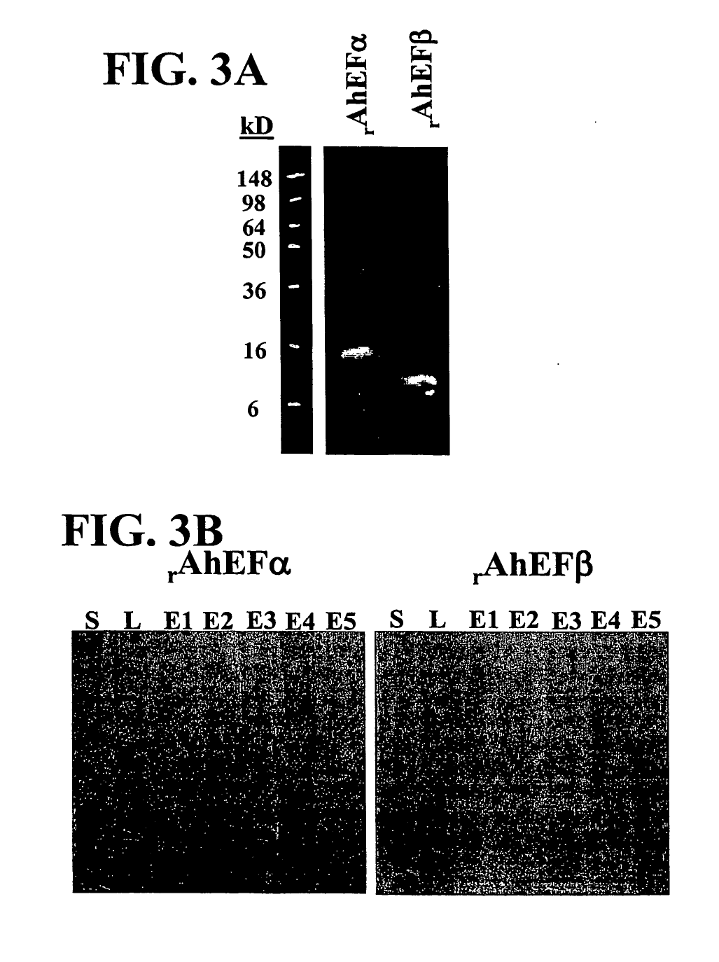

FIG. 3a shows western blots of crude cell lysates containing ᵣAhEFα and ᵣAhEFβ (the expression products of constructs AhT/VD 9 and AhT/VD 22, respectively. Sf21 cells used for transfection were lysed, centrifuged and the resulting supernatants subjected to electrophoresis on 10% polyacrylamide gels. Proteins were transferred to nylon membranes and blots probed with an anti-6×histidine antibody. Following confirmation of ᵣprotein production by western blot analysis, Sf21 cell lysates containing the 2 ᵣproteins were passed through 6×histidine-binding columns, and the bound ᵣproteins eluted in 5 successive 1-ml fractions.

FIG. 3b shows SDS-PAGE of crude lysate (L) and the five 1-ml elutions (E1-E5), stained with Ponceau S. In both cases E3 contained the most purified ᵣprotein. Molecular weight standards on all gels are as follows (from top down: 148 kD, 98 kD, 64 kD, 50 kD, 38 kD and 16 D).

Northern blot analysis was performed using the AhT/VD 9 and Aht/VD 22, respectively, clones. Radio-labelled clone Aht/VD 9 PCR product was used to probe 3 μg/lane of total RNA from the following tissues: fed salivary gland (SG), fed salivary gland (SG), fed testis/vas deferens(F) and unfed testis/vas deferens(U). The same procedure was repeated using PCR product of clone AhT/VD 22 as a probe. Total RNA from each source was electrophoresed on 1.0% agarose-formaldehyde gels and subsequently transferred to nylon membranes. 18S ribosomal RNA was used as a loading standard.

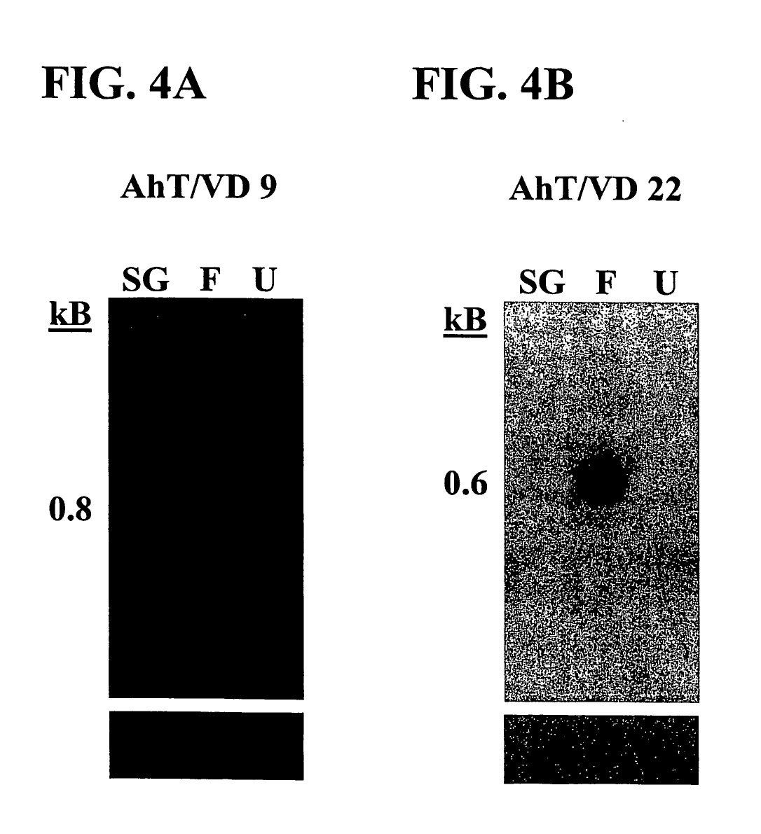

FIG. 4a is a Northern blot analysis of total RNA from fed salivary glands (SG), fed testis/vas deferens(F) and unfed testis/vas deferens(U) when probed with radio-labelled clone AhT/VD 9 PCR product. It can be seen that mRNA for the respective protein was greatly enhanced in fed testis/vas deferens(F).

FIG. 4b is a Northern blot analysis of total RNA from fed salivary glands (SG), fed testis/vas deferens(F) and unfed testis/vas deferens(U) when probed with radio-labelled clone AhT/VD 22 PCR product. It can be seen that RNA for the respective protein was greatly enhanced in fed testis/vas deferens(F).

### EXAMPLE 4

### Engorgement Factor Bio-Assay

Unfed virgin females were placed on rabbits along with a number of fed males which had their gonophores blocked with a small drop of cyanoacrylate glue. The presence of fed males strongly induces females to attach. Females were allowed to feed for 7 days, at which point they are all below the CW (−250 mg in A. hebraeum). Individuals were divided into the treatment groups shown in table 1 and identified by coloured thread tied to a leg segment. All injections were made into the haemocoel via a coxal leg segment, using a 30-gauge needle attached to a Hamilton microlitre syringe. Following injection, ticks were allowed up to 14 days to feed on fresh rabbits (except in the initial experiment (FIG. 3a) in which only 7 days were allowed). During this time any engorged females were weighed, and stored in the colony incubator. All ticks still attached at 14 days were removed, weighed and stored in the colony incubator.

Following removal, some ticks were dissected at 4 days to measure SG degeneration and others at day 10 to measure ovary development. SG degeneration was determined by measuring rate of fluid secretion in vitro as described by Harris and Kaufman (1984). Ovary development was assayed by ovary weight, and compared to data reported for normally engorged females by Friesen et al. (Friesen, K. J., Kaufman, W. R. (2002). Quantification of vitellogenesis and its control by 20-hydroxyecdysone in the ixodid tick, Amblyomma hebraeum, J. Insect Physiol. 48: 773-782), incorporated herein by reference.

### Bioassay of Crude T/VD Homogenates

A partially purified tissue extract of EF was prepared as follows. T/VD of fed males were dissected, homogenized (using glass tissue homogenisers) in chilled saline (1.2% NaCl: 7.5 μl per T/VD) and centrifuged at 8,000 g for 5 min at 4° C. The pellet was discarded and the supernatant stored frozen at −80° C. until required for injection. Partially fed females (all below the CW) were injected with several doses of the partially purified T/VD extract. Control groups were injected with nothing, or 1.2% NaCl, or with 1 accessory gland equivalent from a fed male, or 1 with T/VD equivalent from an unfed mate. Injected females were applied to a fresh rabbit and checked regularly over the next 7 days.

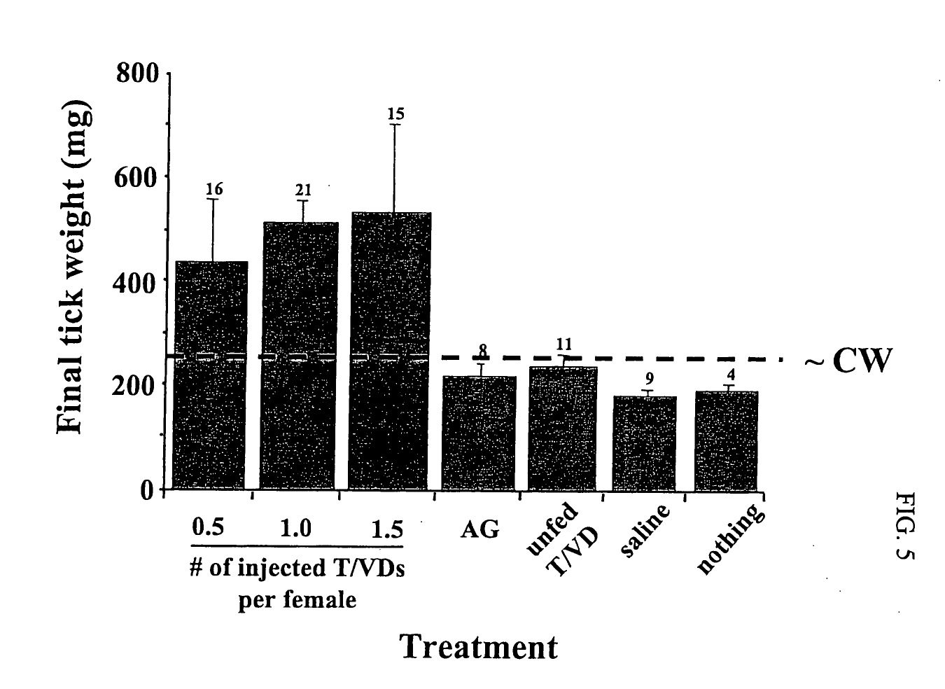

FIG. 5 shows the results when the EF bioassay was performed using crude homogenates made from the T/VD of fed males. Virgin females injected with all three doses (0.5, 1.0 and 1.5 equivalents) of T/VD homogenate fed to significantly above the CW (˜250 mg indicated by dashed line) after being allowed to feed on fresh hosts for seven days. However, those females injected with homogenates of T/VD from unfed males (1 equivalent) or fed accessory gland (1 equivalent) remained below the CW. Uninjected controls or those injected with 1.2% NaCl also remained below the CW.

### Bioassay of the 28 ᵣProteins

The 28 ᵣproteins were initially divided arbitrarily into 2 groups, each containing 14 ᵣproteins. Ticks were injected with one or the other group, but EF bio-activity was not detected in either. This negative result suggested that at least two proteins were necessary for EF bio-activity, one of them being among ᵣproteins 1-14 and the other being among ᵣproteins 15-28. Subsequent groupings of ᵣproteins were tested in order to eliminate those without EF bio-activity. The following control injections were also performed: 1) non-transfected cell lysates, and 2) 5 μg of vector DNA (both pIB/V5-His and pIB/His C). The groupings used, and the bioassay results (which show the mean weight (±SEM) as a function of the indicated treatment), are shown in Table 1.

TABLE 1
Bio-assay of recombinant proteins (ᵣproteins) derived from blood meal-induced mRNA transcripts expressed
in the T/VD of male A. hebraeum.

| | | | mean weight of | mean weight of | fluid secretory | |
|---|---|---|---|---|---|---|
| | | | virgins (mg) at | virgins (mg) at | competence | ovary weight |
| experiment | group # | ᵣproteins | time of injection | detachment by | (mg/gland/15 min) on | (mg) on day |
| # | (n) | injected^{a} | (±SEM) | day 14 (±SEM)^{b} | day 4 post-removal^{c} | 10 post-removal^{d} |
| 1 | 1 (14) | 1-14 | 156 ± 8.9 | 182 ± 7.8 | — | — |
| | 2 (14) | 15-28 | 191 ± 13.3 | 214 ± 6.6 | — | — |
| 2 | 3 (14) | 1-7, 15-20 | 206 ± 5.1 | 211 ± 10.2 | 4.0 ± 0.6 (n = 4) | — |
| | 4 (14) | 1-7, 21-28 | 219 ± 16.1 | 237 ± 10 | 3.9 ± 0.9 (n = 6) | — |
| | 5 (14) | 8-14, 15-20 | 183 ± 11.1 | 194 ± 11.1 | 3.6 ± 0.8 (n = 6) | — |
| | 6 (14) | 8-14, 21-28 | 169 ± 10.1 | 1070 ± 54.8 | 0.4 ± 0.1 (n = 13) | 15.91 ± 1.4 |
| | 7 (7) | control 1 | 219 ± 14.3 | 214 ± 8.8 | 4.2 ± 0.3 (n = 8) | — |
| 3 | 8 (7) | 8-14 | 221 ± 21.0 | 253 ± 8.5 | 4.1 ± 0.3 (n = 4) | 1.6 ± 0.43 |
| | 9 (7) | 21-28 | 178 ± 18.2 | 199 ± 17.4 | 4.7 ± 0.7 (n = 6) | 1.7 ± 0.47 |
| | 10 (7) | 8-14, 21-24 | 236 ± 16.4 | 1651 ± 159 | 0.4 ± 0.1 (n = 10) | 18.12 ± 1.8 |
| | 11 (7) | 8-14, 25-28 | 200 ± 28.1 | 208 ± 18.2 | 3.7 ± 0.5 (n = 4) | 2.0 ± 0.47 |
| | 12 (7) | control 2 | 207 ± 22.3 | 227 ± 12.9 | 4.5 ± 0.4 (n = 8) | 2.1 ± 0.17 |
| 4 | 13 (7) | 8-10, 21, 22 | 185 ± 11.7 | 1979 ± 210 | 0.3 ± 0.1 (n = 8) | 12.5 ± 1.6 |
| | 14 (7) | 11-14, 21, 22 | 202 ± 20.9 | 221 ± 17.2 | 4.7 ± 0.5 (n = 4) | 1.6 ± 0.44 |
| | 15 (7) | 8-10, 23, 24 | 245 ± 22.7 | 194 ± 16 | 4.5 ± 0.3 (n = 4) | 1.8 ± 1.3 |
| | 16 (7) | 11-14, 23, 24 | 192 ± 17.2 | 210 ± 15.7 | 4.0 ± 0.4 (n = 4) | 1.4 ± 0.22 |
| 5 | 17 (7) | 8, 21 | 183 ± 14.8 | 234 ± 23.1 | | |
| | 18 (7) | 8, 22 | 214 ± 15.1 | 206 ± 13.4 | | |
| | 19 (7) | 9, 21 | 170 ± 26.4 | 206 ± 8.2 | | |
| | 20 (7) | 9, 22 | 191 ± 22.9 | 1508 ± 81.0 | | |
| | 21 (7) | 10, 21 | 241 ± 12.5 | 202 ± 9.3 | | |
| | 22 (7) | 10, 22 | 139 ± 9.3 | 230 ± 12.2 | | |

^{a}Control 1 = non-transfected cell lysates: control 2 = 7.5 μg vector DNA (equal to amount used for transfection reactions).
^{b-d}The value of all parameters measured (b-d) for groups (6, 10, 13 and 20) injected with ᵣAhEF was significantly higher (P < 0.0001 in all cases, ANOVA) then the same values for groups not injected with ᵣAhEF.

As can be seen from the results presented in Table 1, the combination of AhT/VD 9 and AhT/VD 22 recombinant proteins gave rise to a significant increase in the mean weight (more than 6 fold) of virgin ticks at detachment by day 14. Such a rise in mean weight only occurred when these two proteins were present in the mix of proteins injected.

### Bioassay of Purified _{c}AhEF.

The two ᵣproteins necessary for EF bio-activity were purified from cell lysates as described under Example 3.

A dose response curve of the two proteins was performed (0.0-1.0 μg of each ᵣprotein) using the EF bioassay. The two controls used were 1) normally-mated females and 2) normally-mated females receiving 7.5 μl of 500 mM imidazole (a potentially toxic antifungal agent found the 6×histidine binding-column elution buffer).

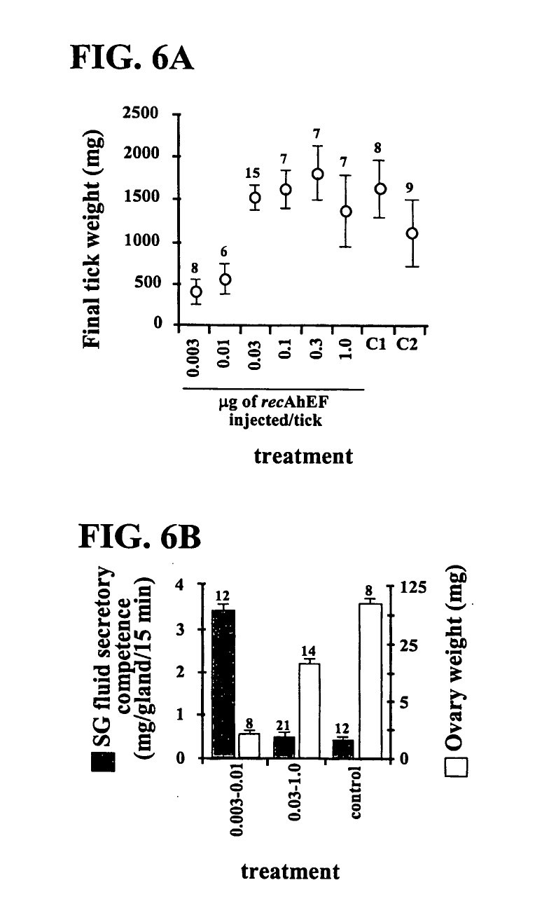

FIG. 6a shows the dose response curve when ticks were injected with purified ᵣAhEF. Virgin females that were injected with 0.03-1.0 μg of pure ᵣAhEF fed to healthy engorged weights, while 0.01 and 0.003 μg of pure ᵣAhEF were unable to stimulate a similar response. One can also see in FIG. 6b that those virgin females that were injected with 0.03-1.0 μg of pure ᵣAhEF also underwent a significant degree of SG degeneration and ovary development. SG degeneration and ovary development did not occur in their counterparts that were injected with the lower doses of ᵣAhEF. Controls in each of FIGS. 6a and 6b are: C1, normally mated females, and C2, normally mated females injected with 500 mM imidazole.

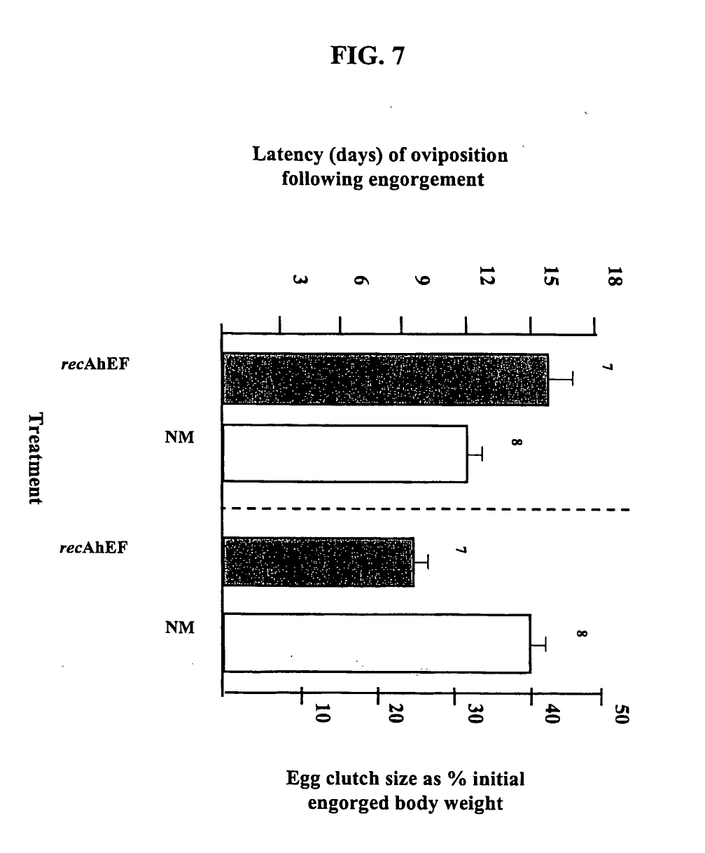

In summary, the data presented in Table 1 and FIG. 6a indicate that ᵣAhEF is able to induce SG degeneration, however, on its own cannot stimulate a full degree of ovary development (Table 1, FIG. 7 and FIG. 6b). Thus, whereas mean ovary weight of virgins injected with ᵣAhEF was 12.5-18 mg 10 days post-engorgement, mean ovary weights of normal mated females of the species is about 104 mg 10 days post-engorgement (Friesen, K. J., Kaufman, W. R. (2002). Quantification of vitallogenesis and its control by 20-hydroxyecdysone in the ixodid tick, Amblyomma habraeum. J. Insect Physiol. 48: 773-782, incorporated herein by reference). Moreover, the latency to oviposition was longer in the engorged virgins displayed in table 1(14-16 days) compared to normal, mated engorged females (−10 days; Friesen, K. J. Kaufman, W. R. (2002). Quantification of vitelogenesis and its control by 20-hydroxyecdysone in the ixodid tick, Amblyomma hebraeum. J. Insect Physiol. 48: 773-782) and the total egg mass was significantly less then that laid by normal engorged females (25% of initial engorged weight vs. 40% respectively). Neither ᵣAhEFα or ᵣAhEFβ on its own, nor any of the other 26 ᵣproteins, display EF or MF bio-activity.

### EXAMPLE 5

The effects of ᵣAhEF on egg production in A. hebraeum were also studied. Females injected with ᵣAhEF were monitored to determine 1) the number of days post-engorgement which elapsed before the beginning of oviposition (latency), and 2) egg clutch size. These data were compared to that of normally mated, engorged ticks (Friesen, K. J., Kaufman, W. R. (2002). Quantification of vitellogenesis and its control by 20-hydroxyecdysone in the ixodid tick, Amblyomma hebraeum. J. Insect Physiol. 48: 773-782). FIG. 7 shows an increased latency period of approximately 12 days in those ticks treated with ᵣAhEF as compared to approximately 10 days for normal mated (NM) females. Similarly, egg clutch size was only about 62% that of normal mated females.

### EXAMPLE 6

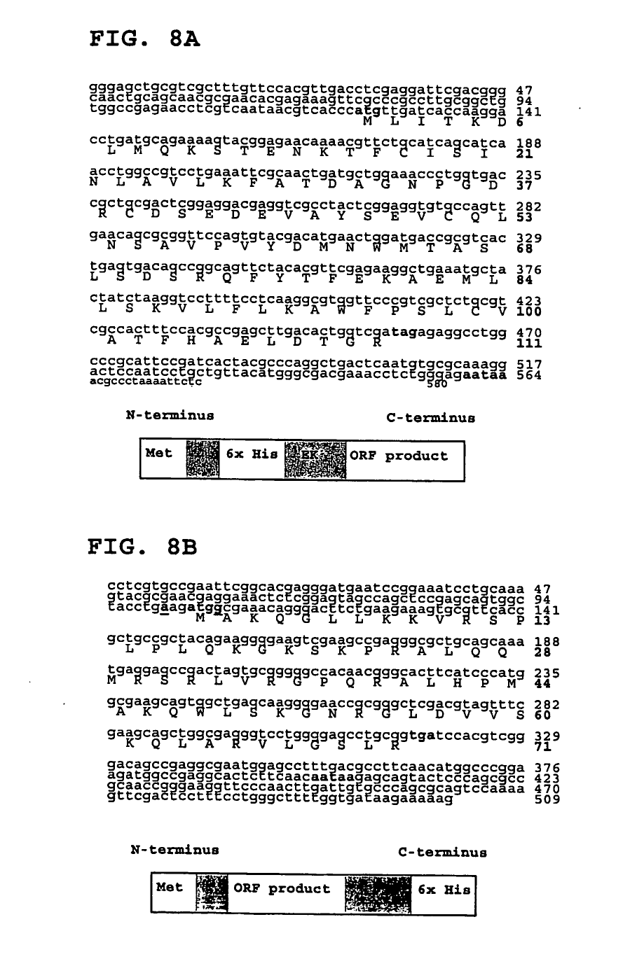

The nucleotide and amino acid sequences of AhT/VD 9 (580 bases) and AhT/VD 22 (509 bases) are shown in FIGS. 8a and 8b, respectively. The start codon (atg), stop codons (tag, tga) and polyadenylation signals are bolded, and the Kozak consensus sequence (in FIG. 8b) is bolded and underlined (Kozak, M. (1990). Downstream secondary structure facilitates recognition of initiator codons by eukaryotic ribosomes. Proc. Natl. Acad. Sci. USA, 87, 8301-8305, incorporated herein by reference).

The upper numbers adjacent to each sequence shown in FIGS. 8a and 8b indicate nucleotide position and bolded numbers indicate amino acid position. Below each nucleotide sequence is a diagrammatic representation of the corresponding ᵣprotein following expression. ᵣAhEFα, which was produced in the plB/His C expression vector, has a N-terminal 6×histidine detection tag. ᵣAhEFβ was produced in the PlB/V5-His expression vector and has a C-terminal 6×histidine detection tag. Shaded boxes represent binding sites for other commercially available antibodies (anti-Xpress and anti-V5 monoclonals; Invitrogen Corp.) spacer regions and an enterokinase cleavage site (EK).

The molecular weight (MW) of native MF, as determined by gel filtration, was reported to be in the range of 20-100 kD (Kaufman, W. R., Lomas, L. O. (1996). “Male factors” in ticks; their role in feeding and egg development. Invert. Repro. and Develop. 30: 191-198). Western blots as shown in FIG. 3a and computer analysis using Peptool software (Biotools Inc., Edmonton, Canada) both indicate that the combined MWs of ᵣAhEFα and ᵣAhEFβ fall within this weight range (˜27.7 kD). This MW is different from tick sperm-capacitation factor (12.5 kD; Shephard, J., et al. (1982). A polypeptide from male accessory glands which triggers maturation of tick spermatozoa. Int. J. Invert. Repro 5: 129-137) and viteliogenesis-stimulating factor (100-200 kD; Connat, et al. (1988). Some aspects of the control of the gonotrophic cycle in the tick. Ornithodoros moubata (Ixodoidea, Argasidae). In: Sauer, J. R., Hair, J. A. (eds.) Morphology, Physiology and Behavioral Biology of Ticks. Ellis Horwood: Chichester), the only two other known mating factors from male ticks. Native EF is like a dimer (possibly larger than 27.7 kD) which, like other male insect sex peptides of similar size (−200-400 amino acids; Monsma, S. A., Wolfner, M. F. (1988). Structure and expression of a Drosophila male accessory gland gene whose product resembles a peptide pheromone precursor. Genes Develop. 2: 1063-1073; Yi, S. X., Gillott, C. (1989). Purification and characterization of an oviposition-stimulating protein from the long hyaline tubules of the male migratory grasshopper, Melanoplpus sanguinipes. J. Insect Physiol. 45: 143-150), may be cleaved into smaller subunits thus making it better able to pass into the female's haemocoel where it presumably has bio-activity.

### EXAMPLE 7

### Active Immunization

To test the tick polypeptides of the present invention for the ability to confer tick immunity, a rabbit was inoculated three times with 150 μg ᵣAhEFα and 150 μg of ᵣAhEFβ at 1-month intervals. The first inoculation was in Freund's complete adjuvant and the other two were with Freund's incomplete adjuvant. One week after the final inoculation, 31 unfed female and 31 unfed male Amblyomma hebraeum ticks were placed on the rabbit in an enclosed arena to feed for up to 14 days. A non-immunized control rabbit was exposed to 25 female ticks (plus males) in the same way.

Turning first to the control rabbit, it was observed that five ticks engorged on day 7, ten on day 8, five on day 8, three on day 10, three on day 11 and two on day 12. Thus, the time to engorgement (mean±SEM) was 8.3±0.3 days (n=28). The average engorged weight was 1899±74 mg. These control ticks laid eggs in the normal way.

When immunized with ᵣAhEFα and ᵣAhEFβ, it was observed that two ticks engorged on day 10, none on day 11, three on day 12, three on day 13 and none on day 14. Average time to engorgement (mean±SEM) was 11.9±0.4 days (n=8). The mean engorged weight of the 8 engorged ticks from the immunized rabbit was 1780±140 mg (n=8) (one of these ticks died a few days after engorgement). The surviving engorged females were all able to lay eggs. On day 14, the remaining 23 partially-fed females were removed and weighed. Average weight was 83±10 mg. Such ticks are much too small to lay any eggs and were much smaller than normal virgin females.

The difference between the engorgement time for the immunized rabbit (11.9±0.4 days) and the control (8.8±0.3 days) was highly significant (p=0.000026; t-test). Further, overall there was a 74% reduction in engorgement success (8/31 engorged vs. 28/28 in control). The average weight of the 8 ticks that did engorge was not significantly lower than that for the normal ticks (p=0.238). The biological significance of the longer time to engorgement (12 days vs. 9 days) among those ticks which did engorge is not entirely clear.

It was surprising that the 23 ticks that failed to engorge were so small. Their average weight was only 83±10 mg after 14 days on a host. We would have hypothesized their average weight to be comparable to that of normal virgin ticks (i.e. on average 198±6.5 mg after 7 days and 213±4.2 mg after 14 days when transferred to a fresh host). Thus, the ticks feeding on the immunized rabbit attained only about 40% the weight expected for normal virgins. One possible explanation is that the antibody to ᵣAhEF is doing more than just inhibiting EF.

Accordingly, the data presented here indicates that immunization with a combination of ᵣAhEFα and ᵣAhEFβ is sufficient to confer tick immunity in an immunized animal.

Using the following formula (PCT Patent Application WO 01/82957, incorporated herein by reference): reduction in average adult female weight=100 (1-(avg. weight of adult females in vaccine group/avg. weight of adult females in control group)), the results showed a 72% reduction in average adult female weight.

1. An isolated nucleic acid comprising a polynucleotide sequence that hybridizes under stringent conditions to a hybridization probe, the nucleic acid sequence of the probe consisting of SEQ ID NO; 1 to the complement of SEQ ID NO:1.

2. A vector comprising the isolated nucleic acid of claim 1.

3. An expression cassette comprising the nucleic acid of claim 1 operably linked to a promoter, wherein the nucleic acid is in sense orientation relative to the promoter.

4. A host cell containing at least one expression cassette of claim 3.

5. An isolated nucleic acid comprising a polynucleotide sequence that hybridizes under stringent conditions to a hybridization probe, the nucleic acid sequence of the probe consisting of SEQ ID NO; 2 or the complement of SEQ ID NO:2.

6. A vector comprising the isolated nucleic acid of claim 5.

7. An expression cassette comprising the nucleic acid of claim 5 operably linked to a promoter, wherein the nucleic acid is in sense orientation relative to the promoter.

8. A host cell containing at least one expression cassette of claim 7.

9. An isolated polypeptide having Engorgement Factor activity, selected from the group comprising:
a) a polypeptide having an amino acid sequence which has at least 80% homology with the amino acid sequence of SEQ ID NO:3;
b) a polypeptide which is encoded by a nucleic acid sequence which hybridizes under stringent conditions with the nucleic acid sequence of SEQ ID NO:1; or
c) a fragment of (a) or (b) that has Engorgement Factor activity.

10. The polypeptide of claim 9, wherein the amino acid sequence of the polypeptide has at least 85% homology with an amino acid sequence of SEQ ID NO:3.

11. The polypeptide of claim 9, wherein the amino acid sequence of the polypeptide has at least 95% homology with an amino acid sequence of SEQ ID NO:3.

12. An isolated polypeptide having Engorgement Factor activity, selected from the group comprising:
a) a polypeptide having an amino acid sequence which has at least 80% homology with the amino acid sequence of SEQ ID NO:4;
b) a polypeptide which is encoded by a nucleic acid sequence which hybridizes under stringent conditions with the nucleic acid sequence of SEQ ID NO:2; or
c) a fragment of (a) or (b) that has Engorgement Factor activity.

13. The polypeptide of claim 12, wherein the amino acid sequence of the polypeptide has at least 85% homology with an amino acid sequence of SEQ ID NO:4.

14. The polypeptide of claim 12, wherein the amino acid sequence of the polypeptide has at least 95% homology with an amino acid sequence of SEQ ID NO:4.

15. A vaccine for reduction of transmission of tick-borne pathogens or tick-borne disease, wherein said vaccine comprises administration of the isolated polypeptide of claim 9 and a pharmaceutically acceptable carrier.

16. A vaccine for reduction of transmission of tick-borne pathogens or tick-borne disease, wherein said vaccine comprises administration of the isolated polypeptide of claim 12 and a pharmaceutically acceptable carrier.

17. A vaccine composition comprising an immunogenic fragment of the polypeptide of SEQ ID NO:3 wherein said immunogenic fragment is in a pharmaceutically acceptable carrier and wherein said immunogenic fragment is present in an amount effective to elicit protective antibodies in a mammal against Engorgement Factor proteins.

18. The vaccine composition of claim 17 wherein the mammal is a human.

19. A vaccine composition comprising an immunogenic fragment of the polypeptide of SEQ ID NO:4 wherein said immunogenic fragment is in a pharmaceutically acceptable carrier and wherein said immunogenic fragment is present in an amount effective to elicit protective antibodies in a mammal against Engorgement Factor proteins.

20. The vaccine composition of claim 19 wherein the mammal is a human.

21. A method for preventing infection by a tick-borne pathogen or a tick-borne disease, comprising administration to a subject a polypeptide according to claim 9.

22. A method for preventing infection by a tick-borne pathogen or a tick-borne disease, comprising administration to a subject a polypeptide according to claim 12.

23. An antibody or an antigen binding portion thereof comprising an antibody or antigen portion thereof capable of specifically binding a polypeptide selected from the group comprising a polypeptide of SEQ ID NO:3 or a polypeptide of SEQ ID NO:4.

24. A method to detect an antibody or antigen binding portion thereof capable of binding to the polypeptide of SEQ ID NO:3 or SEQ ID NO:4 comprising:
a) contacting a sample containing at least one antibody or antigen binding portion thereof with a polypeptide selected form the group comprising the polypeptide of SEQ ID NO:3 and SEQ ID NO:4, under conditions which allow the antibody or antigen binding portion thereof to bind to said polypeptide; and
b) detecting the binding of the antibody to said polypeptide.
